(19) United States
(12) Patent Application Publication
(10) Pub. No.: US 2022/0257430 A1
(43) Pub. Date: Aug. 18, 2022

(54) TUNABLE ABSORBENT UNDERGARMENT

(71) Applicant: The Flex Company, Venice, CA (US)

(72) Inventors: Andrew Ross Miller, Oakland, CA (US); Jane Hartman Adame, Oakland, CA (US); Brittney Seel, Venice, CA (US); Lauren Schulte Wang, Topanga Canyon, CA (US); Desirée Judith CADMAN MENDOZA, San Francisco, CA (US); Nancy Jean BACH, Richmond, CA (US)

(21) Appl. No.: 17/737,790

(22) Filed: May 5, 2022

Related U.S. Application Data
(63) Continuation of application No. PCT/US2020/059197, filed on Nov. 5, 2020.
(60) Provisional application No. 62/930,801, filed on Nov. 5, 2019.

(51) Int. Cl. A61F 13/496; A61F 13/491; A61F 13/535

(52) U.S. Cl. CPC A61F 13/496; A61F 2013/530875; A61F 13/535; A61F 13/491

(57) ABSTRACT

An absorbent undergarment with a tunable absorbent portion. The tunable portion being tunable to absorb and/or capture both fluid materials as well as more viscous, solid, and/or semi-solid materials. Thus, the undergarment affords the user improved wearability as well as comfort and avoids unwanted odors from exposed material.

### CROSS REFERENCE TO RELATED APPLICATION

The present application is a continuation of International Application Number PCT/US2020/059197, filed Nov. 5, 2020, which claims priority to U.S. Provisional Patent Application Ser. No. 62/930,801, filed Nov. 5, 2019, all of which are hereby incorporated by reference in their entirety.

### FIELD OF THE INVENTION

The present invention generally relates to undergarments, more specifically, undergarments that are designed to absorb bodily fluids such as those associated with menstruation and incontinence.

### BACKGROUND OF THE INVENTION

A person who menstruates will typically use a tampon or pad to absorb catamenial fluids. Alternatives to tampons and pads exist to collect catamenial fluids such as menstrual cups. Meanwhile, undergarments provide several functions such as shaping the wearer's body, supporting the body during exercise, and offering protection to outer clothing. There are undergarments designed to collect fluids such as catamenial fluids or urine and are referred to as period underwear or incontinence underwear respectively as described in U.S. Pat. Pub. 2014/0039432 to Saint ET. Al (Saint) and CN 10,231,885 to Hovey (Hovey).

Period underwear or absorbent undergarments have been developed and marketed as a product that can replace the use of tampons or absorbent pads. The development of reusable materials and products for the absorption of bodily fluids is a growing field because their use can lead to reduced waste and lessen the impact on the environment. While some absorbent undergarments can be worn as a primary product, some are often worn in conjunction with other menstrual care products to help manage any leaks. Traditional absorbent undergarments rely on manufacturing methods that consist of multiple absorbent and/or hydrophobic layers to absorb fluids and prevent leaks. Such traditional methods of manufacture only vary slightly between manufacturers and often lead to increased thickness for added absorbency that subsequently leads to user discomfort and dissatisfaction.

### BRIEF SUMMARY OF THE INVENTION

Many embodiments are directed to undergarments with improved absorption capabilities that may be designed to offer increase absorption of less viscous materials as well as the capture of more viscous, sold, and semi-solid material. Thus, drawing such thicker substances away from the body to reduce odor and user discomfort.

Many embodiments include an absorbent undergarment that has:
a body of material having a first opening on an upper end of the body and two lower openings defining a central section therebetween on a lower portion of the body, wherein the central section further comprises a plurality of material layers configured to absorb viscous and fluid material;
wherein at least one layer of the plurality of layers of material is configured to control a flow of viscous material and comprises a plurality of protrusions wherein each of the plurality or protrusions are separated by a space such that the flow of viscous material can be directed to and captured within the space, wherein the at least one layer of plurality of layers is further configured to absorb fluid material, and
wherein the plurality of protrusions and spaces may be configured to adjust the absorption tunability of the viscous and fluid material of the undergarment.

In other embodiments, the plurality of protrusions extend outward from a first surface of the at least one layer.

In yet other embodiments, the plurality of protrusions are configured in such as way so as to make up an entire thickness of the at least one layer.

In still other embodiments, the plurality of protrusions are disposed in such a manner that the protrusions form a plurality of diamond shaped structures within the outer surface of the at least one layer and wherein the spaces form a plurality of channels between the plurality of diamond shaped structures.

In still yet other embodiments, the plurality of protrusions are disposed in such a manner that the protrusions form a plurality of circular shaped structures within the outer surface of the at least one layer and wherein the spaces form a plurality of channels between the plurality of diamond shaped structures.

In other embodiments, the plurality of protrusions are disposed in such a manner that the protrusions form a plurality of amorphous shaped structures within the outer surface of the at least one layer and wherein the spaces form a plurality of channels between the plurality of diamond shaped structures.

In yet other embodiments, each of the plurality of protrusions is configured to fold over towards an adjacent protrusion.

In still other embodiments, each of the protrusions extend 0.1 mm to 6 mm from the first surface.

In still yet other embodiments, each of the plurality of protrusions has a width of 0.1 mm to 7 mm when viewed from the side.

In other embodiments, the at least one layer further comprises a plurality of fenestrations that extend through at least a portion of the at least one layer and may be configured to control the flow of viscous and fluid material through the at least one layer.

In yet other embodiments, the fenestrations are disposed within the spaces.

In still other embodiments, the plurality of fenestrations are disposed within the protrusions.

In still yet other embodiments, the plurality of fenestrations extend through an entire thickness of the at least one layer.

In other embodiments, the at least one layer is an outer most layer of the center section.

In yet other embodiments, the at least a second material layer is configured to control the flow of the viscous and fluid material.

In still other embodiments, the at least second layer comprises a plurality of fenestrations disposed within the at least second layer and extending at least through a portion of the at least second layer.

In still yet other embodiments, the plurality of fenestrations extend through an entire thickness of the at least second layer.

In other embodiments, the undergarment has a plurality of protrusions wherein each of the plurality or protrusions are separated by a space such that the flow of viscous material can be directed to and captured within the space, wherein the at least one layer of plurality of layers is further configured to absorb fluid material, and wherein the plurality of protrusions and spaces may be configured to adjust the absorption tunability of the viscous and fluid material of the undergarment.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates a layered absorption system in accordance with known art.

FIGS. 2A and 2B illustrate an absorption system in accordance with embodiments of the invention.

FIG. 3 illustrates an absorption system with a textured surface in accordance with embodiments of the invention.

FIGS. 4A through 4C illustrate cross sectional patterns of absorption layers in accordance with embodiments of the invention.

FIG. 5 illustrates an absorbent undergarment in accordance with embodiments of the invention.

FIGS. 6A through 7B illustrate various patterns of absorption layers in accordance with embodiments of the invention.

FIGS. 8A through 8C illustrate fiber patterns and cross sections that be utilized in accordance with embodiments of the invention.

### DETAILED DESCRIPTION OF THE INVENTION

Turning now to the drawings, systems and methods for improving the absorption capabilities of an absorbent undergarment are illustrated. Many embodiments include an absorbent undergarment with a body having a front and a back portion where an opening for a waist is defined between the front and back portions. Additionally, a pair of openings for legs that define a pubic area region therebetween is positioned beneath the waist opening. The pubic area region is further defined as having a moisture absorbent and leak resistant section that covers at least a portion of the pubic area region. In some embodiments, the moisture absorbent and leak resistant section can cover a portion of the front part and/or the back part of the garment. In many embodiments the moisture absorbent and leak resistant section has a tunable primary absorbent surface that can absorb less viscous fluid while capturing viscous, solid, and semi-solid fluid components.

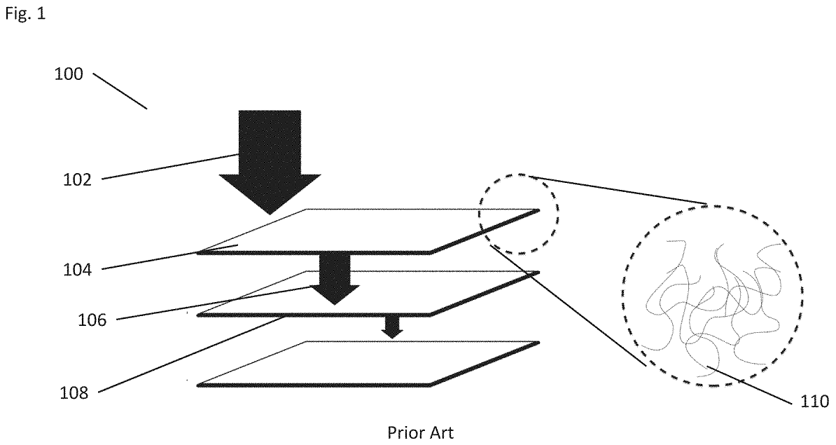

Absorbent undergarments are traditionally made with multiple layers of material. For example, the main body of the undergarment can have one or more layers of material that are used to form the main body. Additionally, typical absorbent undergarments usually have multiple layers of material placed in a pubic area region designed to absorb bodily fluids. The traditional form of layers often includes two or more layers including an absorbent layer and a hydrophobic layer in addition to the undergarment material itself. One major limitation of the known art is the amount of fluid that can be absorbed. This is often due to the traditional manufacturing methods that require more layers of absorbent material in order to allow for more absorption. Accordingly, the typical design of absorbent undergarments in not capable of extended use beyond a couple of hours. Extended use can lead to leaking and general user discomfort and dissatisfaction. One solution that is often applied is to use additional layers of material stacked in a laminate form of construction. However, the laminate form of construction does not always provide for greater absorbency. For example, as illustrated in FIG. 1, only a portion of the main fluid 102 may pass through the first layer 104. Likewise, a smaller amount of the absorbed fluid 106 would only be flow through the second layer 108. This is regardless of the type of absorbent material 110 that is used. This reduction in flow would only continue as additional layers are added. Thus, not only would the additional layers add unwanted bulk to the undergarment, they would not necessarily allow for increased absorption, leading to potential leaks.

Furthermore, such laminate construction of more planar type materials tend to only be useful for lighter flow days for the user because of limited total fluid absorption and more viscous material from the users flow tends to remain on the surface of the undergarment rather than be absorbed. Consequently, the remaining material has a greater chance of oxidation which results in unwanted odor and increased user discomfort. Moreover, many of the materials used in these multilayer laminates are not suitable to be machine dried. Therefore, traditional absorbent undergarments can be inconvenient to use over the length of a menstrual cycle.

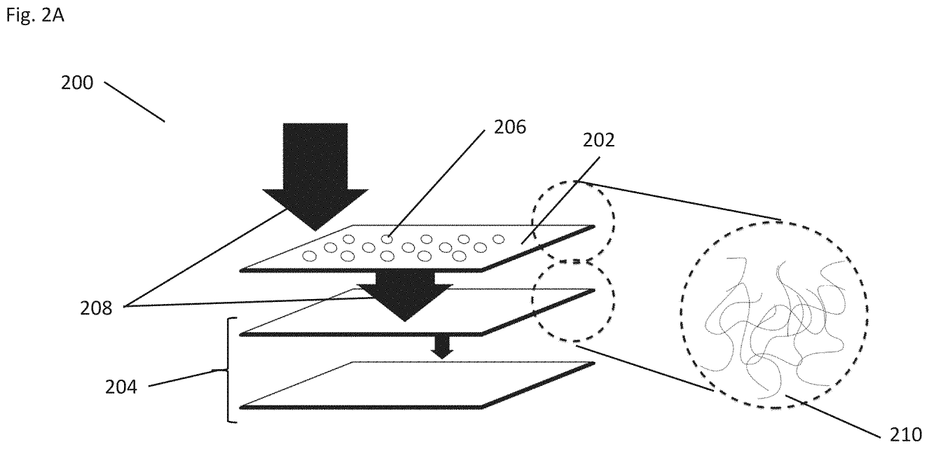

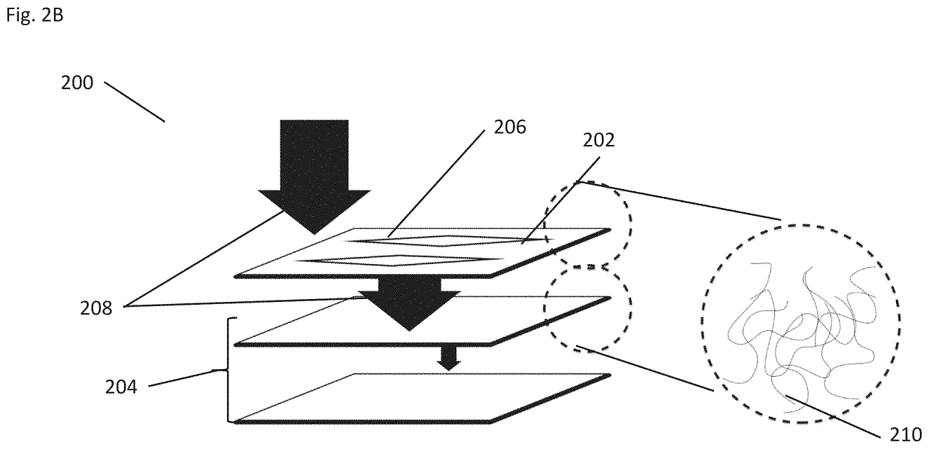

In accordance with many embodiments, laminate type construction can be used with a tunable upper surface that allows for improved absorption into the lower layers as well as provides systems and methods to capture the more viscous, solid, and semi-solid material away from the user and thus preventing unwanted odor. For example, FIGS. 2A and 2B illustrates a laminate structure 200 in accordance with embodiments that has a tunable outer layer 202 and additional lower layers 204 positioned beneath the outer tunable layer. The outer layer 202 can be one that is positioned next to or closest to the user's body. In some embodiments the outer layer 202 can be formed as a distinct section of the main material used in the undergarment or may be a separate layer of material.

In accordance with many embodiments, the outer layer 202 may be tunable to allow for improved flow of the bodily fluids. For example, in some embodiments the upper layer may have a number of fenestrations 206. The fenestrations 206 may be configured in a number of sizes and shapes and may take on any number of patterns within the outer layer 2020. Fenestrations 206 can improved the flow and absorption of the less viscous by allowing quicker movement of larger amounts of material to the lower absorption layers, as illustrated by the similar sized arrows 208. Additionally, the fenestrations 206 can allow more viscous, solid, and semi-solid material to pass to lower layers 204 such that they are not in contact with the body, thereby helping to reduce unwanted odor that can occur from extended exposure of the more viscous fluids. In accordance with many embodiments, the fenestrations 206 may come in a number shapes and sizes, as further illustrated in FIG. 2B. This adjustability can provide for a more tunable layer thereby improving the absorbency of the layer and the fluid and solid transfer of materials from one layer to the next. In accordance with many embodiments the absorbent material 210 of the layers can take on any number of forms and/or configurations and use any number of different materials that may be sufficient to provide enough absorption. Although the upper most layer is illustrated as being tunable, it should be understood that many embodiments may incorporate multiple tunable layers throughout the undergarment which can allow for improved flow and absorption in the undergarment thereby allowing for extended use.

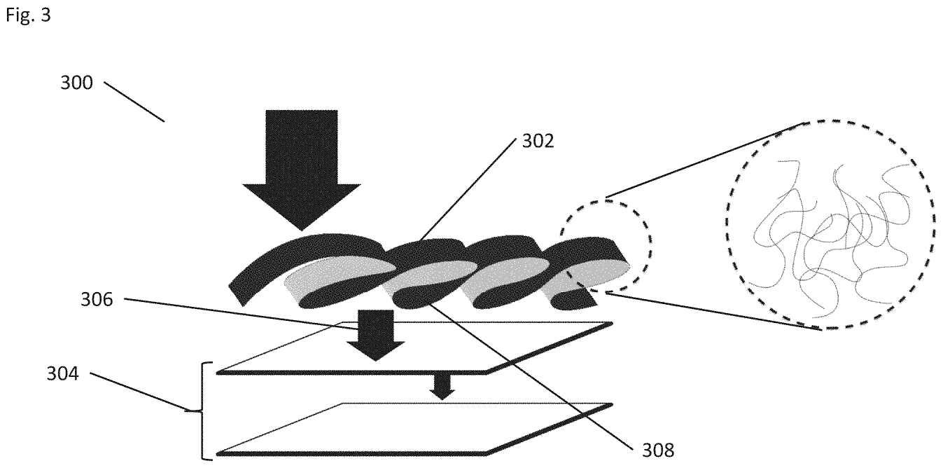

Turning now to FIG. 3, a laminate structure 300 of an absorbent material in accordance with embodiments is shown. In a number of embodiments, the laminate structure can utilize a texturing 302 on the outer most layer of material or the layer that comes in contact with the user's body. The texturing 302 can be a three dimensional textured surface that operates to improve absorption of the bodily fluids and also provide a catchment ability that can draw more viscous fluid away from the body. Since absorbency is a function of surface area of the material, the absorbency of the material will increase linearly with the increased surface area exposed to the fluid. Accordingly, a textured surface 302 applied to the outer layer of material in various embodiments can operate to absorb more fluid in the outer layer than more traditional planar material. Subsequently, the lower layers 304 can be configured to absorb more fluid. In addition to the improved flow capabilities of increased surface area, the textured surface can also provide pockets 308 for capturing the viscous, sold, and semi-solid material to keep it away from the body; thus, preventing unwanted odor. The pockets 308 in conjunction with the raised or textured portion 302 can act as sealed sections that lock the viscous fluid away from the body, thus allowing for more extended use.

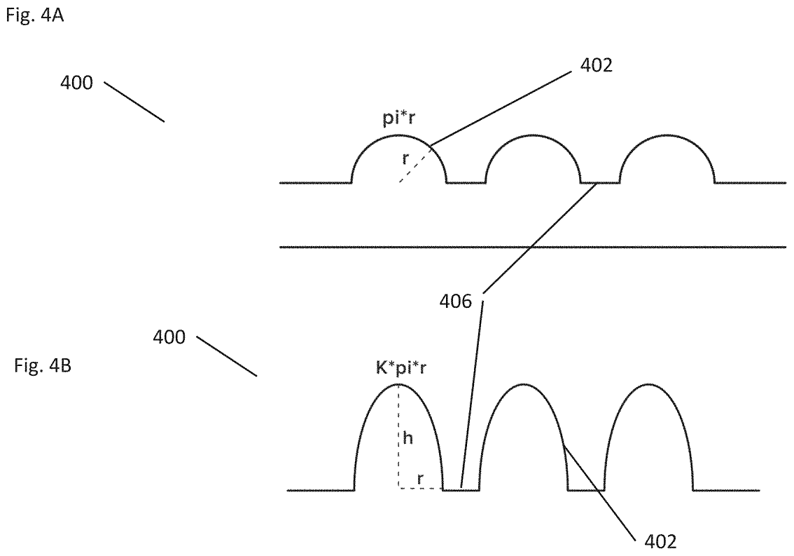

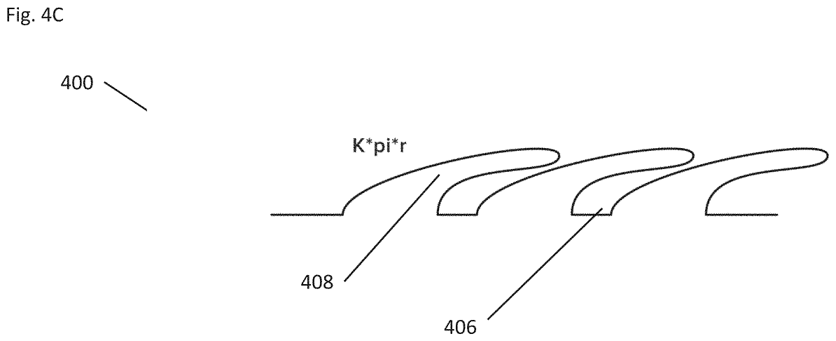

It can be readily appreciated that the textured layers and configurations can be used in any number of layers to provide improved absorbency capabilities of the undergarment. For example, in some embodiments only the top layer may be textured while other embodiments may apply a form of texturing to all layers or alternating layers within the undergarment. Furthermore, it can be appreciated that the texturing or protruding elements of the layer of material can be present throughout the thickness of the layer or may be present in only a portion of the layer. For example, the texture material in FIG. 3 is illustrative of an entire layer thickness having texturing features, while FIGS. 4A through 4C illustrate embodiments where a surface portion of the layer contains the texturing elements. It can be appreciated that such elements can allow for an increased range of consumer products that more closely match the needs of many different consumers.

In accordance with many embodiments, the textured surface can have any type of cross section and can be configured in any desirable dimension. For example, FIGS. 4A through 4C illustrate cross sectional views of a textured surface that can be used in any number of embodiments. FIGS. 4A and 4B illustrate cross sections of a textured absorbent layer of material 400 having one or more protrusions 402. In between each of the protrusions or raised portions 402 may be a gap or pocket 406 that creates a valley type structure. The gap or pocket 406 can act as a catchment area for the outer layer that can catch and/or direct fluid. For example, the more viscous, solid, and semi-solid material may flow more easily into the pockets 406 and can be directed away from the user's body thereby preventing the unwanted odor that can often occur. Additionally, as will be illustrated further, many embodiments of the textured material can configure the pattern of the protrusions such that the pockets or valleys 406 form channels that can direct or catch the fluid. In a number of embodiments, the channels or valleys 406 can also be configured with additional folds and/or fenestrations to allow for improved flow within the body of the undergarment. In accordance with many embodiments the protrusions can extend beyond the surface to any desirable distance such as from 0.1 mm to 6 mm or may be more or less depending on the desired functionality. Additionally, the cross sectional width of each of the protrusion can vary between 0.1 mm to 7 mm in width.

FIG. 4C illustrates a cross section of a textured absorbent layer where the textured surface is represented by a series of folds 408. Similar to other embodiments, the folds increase the surface area of the absorbent layer, however, the use of folds help to reduce the overall thickness of the layer since the folds 408 may not have the same height as other protrusions. The reduced thickness can therefore improve user comfort. Additionally, the folds 408 may incorporate similar gaps 406 as other cross sections, but the fold can help to provide further separation between the capture material and the body, thereby reducing unwanted order.

Although certain cross sections are illustrated it should be understood that the textured surface may take on any type of cross section. For example, many embodiments may have a number of one or more of the following features to help improve the absorbent characteristics of the layer;
Pleats;
Fenestrations;
Wrinkles;
Pits;
Pockets;
Channels;
Grooves (random or organized); and
Strips of fabric sewn together to make a sandwich of permeable layers.

Additionally, the method of texturing can come from any number of desirable methods including, molding, sewing, 3-D knitting, 4-D knitting, and/or a combination of such methods. In accordance with many embodiments, the textured surface may have layers below and above that are formed in the same pattern to help improve the overall function, appearance, and/or comfort of the absorbent undergarment.

Although certain embodiments of absorbent materials have been illustrated, it should be understood that any combination of the absorbent elements can be used separately or in conjunction with other elements and configurations of absorbent materials within the many embodiments described herein.

### Embodiments of an Undergarment

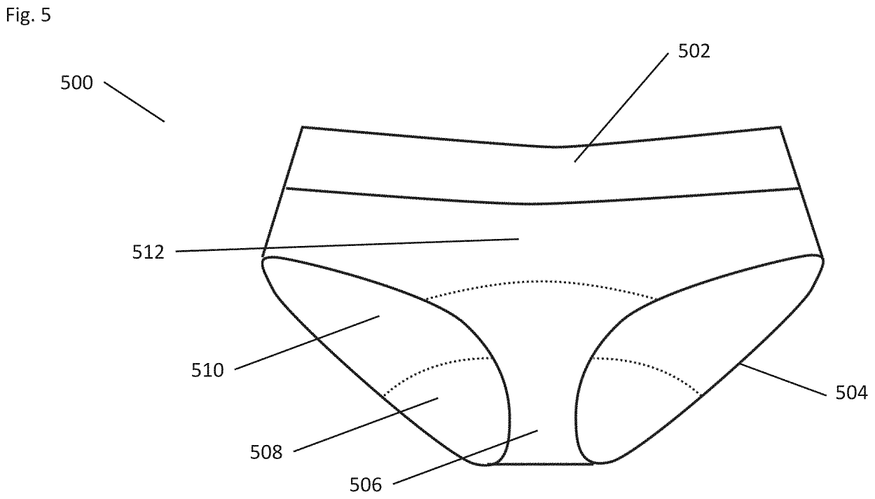

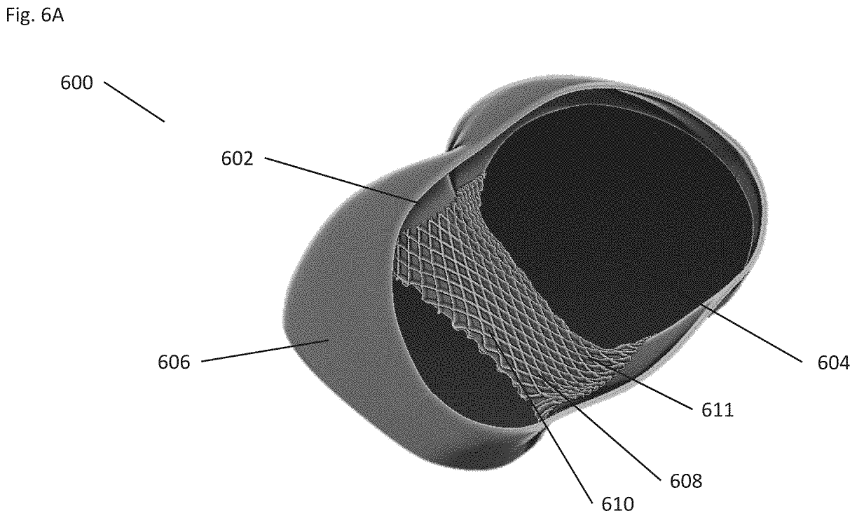

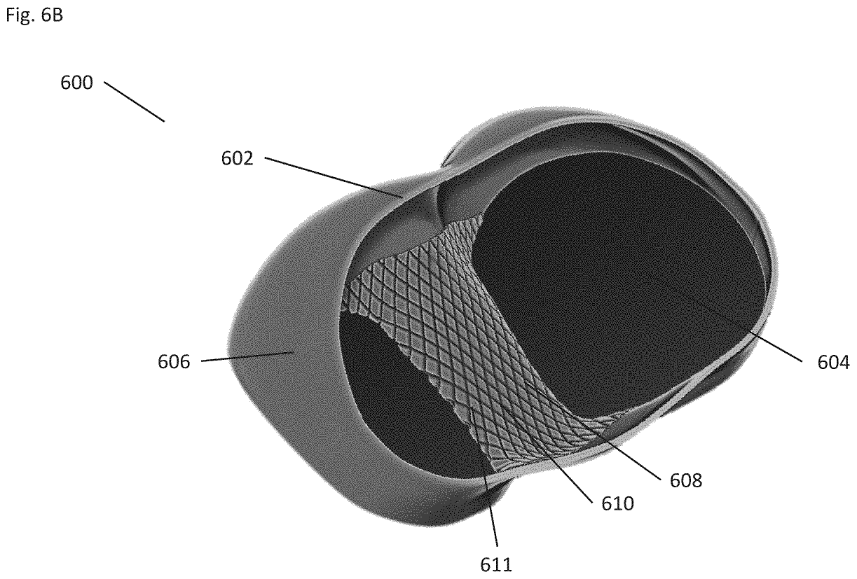

Turning now to FIGS. 5 through 6D, embodiments of undergarments with absorbent portions are shown. FIG. 5 illustrates the basic layout of an undergarment 500 with a section designed to be more absorbent. As with many traditional undergarments, the undergarment 500 can have a waist portion 502 and two leg portions 504. Although not seen in this view, the waist portion 502 has an opening. Additionally, a gusset 506 of the undergarment 500 can be specifically designed to be an absorbent gusset section of the undergarment 500 that has one or more layers of absorbent material 508. In accordance with many embodiments, the absorbent material 508 may occupy all or some of the gusset portion 506 as well as some portion of the front and back portions (510 & 512) of the undergarment 500. Although a specific configuration is illustrated, it should be understood that the basic structure of the undergarment 500 can be modified in any particular manner so as to be suitable for the desired use.

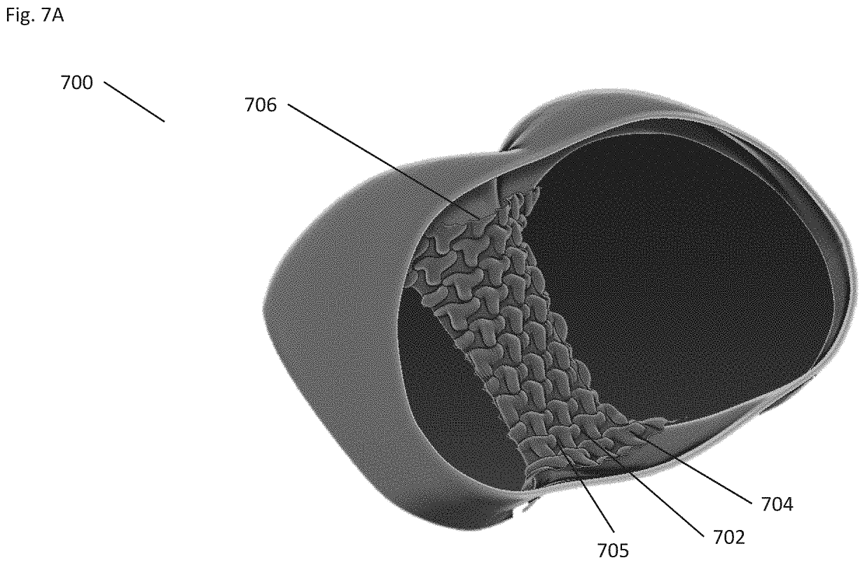

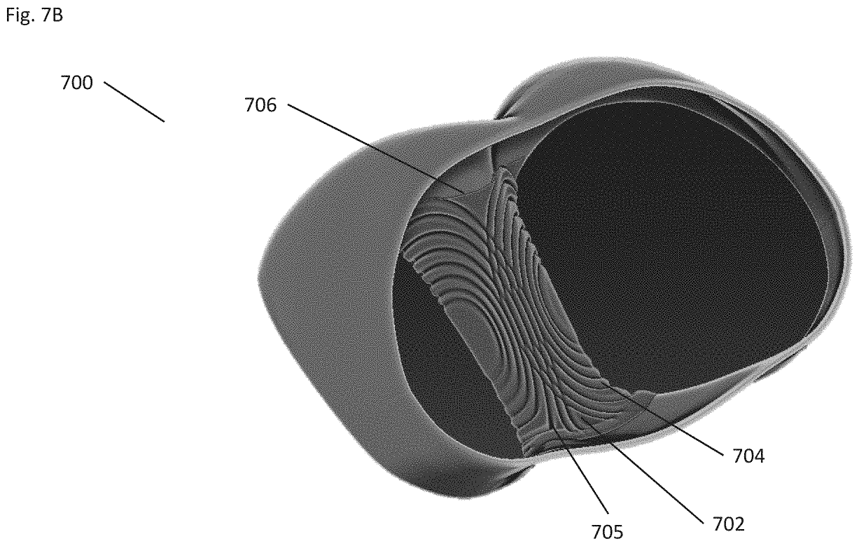

Similar to FIG. 5, FIGS. 6A through 7B illustrate an absorbent undergarments 600 each with a waist opening 602 and leg openings 604. The body of the garment 606 can be more fully appreciated with the perspective views illustrated in FIGS. 6A through 6D. Additionally, FIGS. 6A through 6D illustrate a variety of textured patterns 608 in the gusset section of the undergarment. As described earlier, textured patterns can be the result of the textured surface layer of the undergarment and can serve to provide a more absorbent portion of the undergarment through a number of different methods. As can be appreciated the textured patterns 608 can take on any number of forms that can serve a number of different purposes beyond just absorption. For example, the textured pattern 608 can have a diamond shaped element 610 that leaves corresponding lines 611 between the respective diamond elements 610. Accordingly, some embodiments can be configured to use the lines 611 to direct the flow of fluid or trap the fluid in a particular area and away from the body. Additionally, the diamond portion may be raised while the lines are depressed or the opposite may be the case as illustrated between FIGS. 6A and 6B. Accordingly, some embodiments may utilize raised lines with diamond shaped fenestrations to allow for improved flow and absorption characteristics of the outer layer.

The raise/depressed sections can take on any number of different patterns or shapes as further illustrated in FIGS. 7A and 7B. The textured pattern 702 of the undergarment 700 can have raised 704 and depressed 705 sections that make up the pattern. The raised portions 704 can be configured similar to the various cross sections illustrated above having protrusion and or folds build within the raised portion 704 to improve absorption. Similarly the depressed portion 705 can have micro protrusions to help for absorption and flow of that area of the textured layer 702. Moreover, although a certain configuration of raised and depressed sections is illustrated, it can be appreciated that the raised 704 and depressed 705 sections can be flipped in orientation in a number of embodiments. Accordingly, some sections may have fenestrations as a portion of the depressed 705 sections to improve flow and capture viscous, solid, and semi-solid material. Additionally, similar to the raised and depressed sections described above, the various pattern in the gusset region 706 can act as a flow director or control method to help prevent leaks outside of the gusset region 706. As can be appreciated, the number and variety of texture elements can vary greatly and that any number of combinations can be used. As illustrated in FIG. 7A an amorphous type configuration of texturing elements can be used. Likewise, FIG. 7B illustrates a more defined overlapping circular pattern of texturing elements.

Although certain embodiments of absorbent materials and/or textured patterns within an absorbent undergarment have been illustrated, it should be understood that any combination of the absorbent elements and/or textured patterns can be used separately or in conjunction with other elements, configurations, and/or textured patterns of absorbent materials within the many embodiments described herein.

### Embodiments of Materials

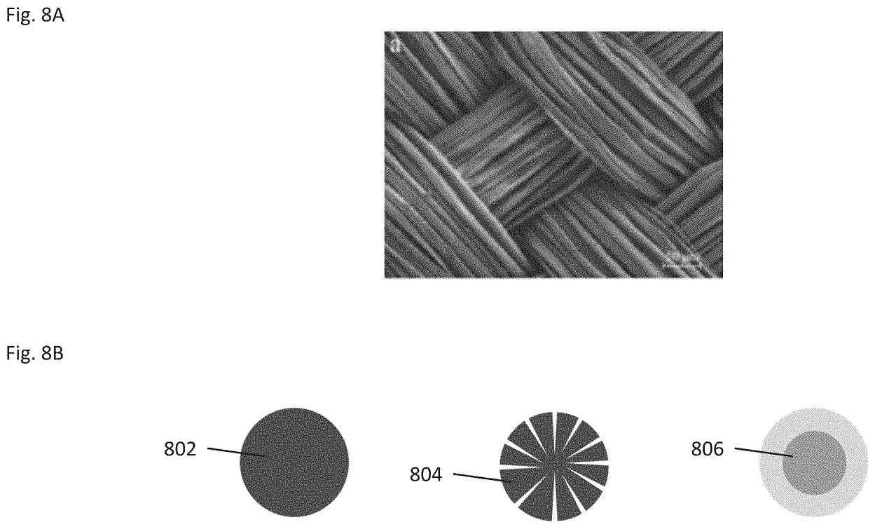

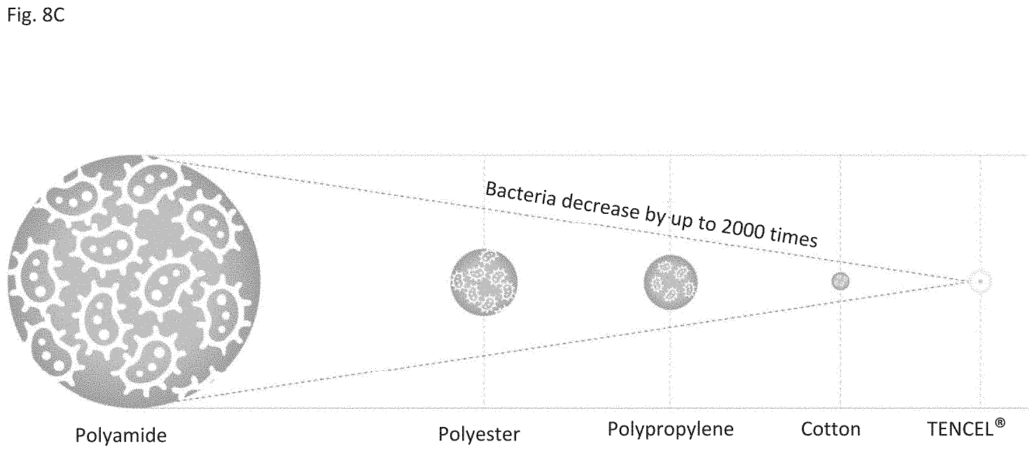

As can be appreciated through the various embodiments of textured patterns and configurations absorption capabilities can also be based on material, material structure as well as material pattern. For example, FIGS. 8A through 8C illustrate various types of fibers and fiber patterns that may be used within the absorbent layer in accordance with embodiments of the invention. FIG. 8A, for example, illustrates an example of a fiber pattern 800 that has multiple diameters of fibers creating multiple spaces that can perform functions of both wicking and drying. Thus, many embodiments may incorporate a predesigned knit pattern to improve the absorbency of the layer. Likewise, the fiber itself can aid in the absorbency function. For example, FIGS. 8B and 8C illustrate the differences between a Polyamide microfiber and a biodegradable Tencel fiber. Most traditional absorbent undergarments use a microfiber material that may be either solid 802 or cracked 804. The Polyamide microfiber material, both cracked and un-cracked, can allow for good absorption properties due to the increased surface area. However, cracked fibers have a tendency to absorb bacteria in addition to fluids. Tencel fibers, alternatively will absorb fluid and not bacteria. Additionally, Tencel fibers can retain their mechanical properties when wet. Furthermore, Tencel fibers offer improved durability (wash and dry) as well as less of an environmental impact. Although certain fibers are illustrated, it should be understood that the absorbent layers may be made up of any number of material and/or fibers that provide good absorbent properties.

### DOCTRINE OF EQUIVALENTS

As can be inferred from the above discussion, the above-mentioned concepts can be implemented in a variety of arrangements in accordance with embodiments of the invention. Specifically, absorbent layers for use in undergarments or the like with tunable surfaces that improve the absorbency of the overall layer and/or product. Achieving such functionality, according to embodiments, involves the implementation of special arrangements/designs between subsystems described above, and their equivalents.

Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. An absorbent undergarment comprising;
a body of material having a first opening on an upper end of the body and two lower openings defining a central section therebetween on a lower portion of the body, wherein the central section further comprises a plurality of material layers configured to absorb viscous and fluid material;
wherein at least one layer of the plurality of layers of material is configured to control a flow of viscous material and comprises a plurality of protrusions wherein each of the plurality or protrusions are separated by a space such that the flow of viscous material can be directed to and captured within the space, wherein the at least one layer of plurality of layers is further configured to absorb fluid material, and
wherein the plurality of protrusions and spaces may be configured to adjust the absorption tunability of the viscous and fluid material of the undergarment.

2. The absorbent undergarment of claim 1, wherein the plurality of protrusions extend outward from a first surface of the at least one layer.

3. The absorbent undergarment of claim 1, wherein the plurality of protrusions are configured in such as way so as to make up an entire thickness of the at least one layer.

4. The absorbent undergarment of claim 1, wherein the plurality of protrusions are disposed in such a manner that the protrusions form a plurality of diamond shaped structures within the outer surface of the at least one layer and wherein the spaces form a plurality of channels between the plurality of diamond shaped structures.

5. The absorbent undergarment of claim 1, wherein the plurality of protrusions are disposed in such a manner that the protrusions form a plurality of circular shaped structures within the outer surface of the at least one layer and wherein the spaces form a plurality of channels between the plurality of diamond shaped structures.

6. The absorbent undergarment of claim 1, wherein the plurality of protrusions are disposed in such a manner that the protrusions form a plurality of amorphous shaped structures within the outer surface of the at least one layer and wherein the spaces form a plurality of channels between the plurality of diamond shaped structures.

7. The absorbent undergarment of claim 1, wherein each of the plurality of protrusions is configured to fold over towards an adjacent protrusion.

8. The absorbent undergarment of claim 2, wherein each of the protrusions extend 0.1 mm to 6 mm from the first surface.

9. The absorbent undergarment of claim 2, wherein each of the plurality of protrusions has a width of 0.1 mm to 7 mm when viewed from the side.

10. The absorbent undergarment of claim 1, wherein the at least one layer further comprises a plurality of fenestrations that extend through at least a portion of the at least one layer and may be configured to control the flow of viscous and fluid material through the at least one layer.

11. The absorbent undergarment of claim 10, wherein the fenestrations are disposed within the spaces.

12. The absorbent undergarment of claim 10, wherein the plurality of fenestrations are disposed within the protrusions.

13. The absorbent undergarment of claim 10, wherein the plurality of fenestrations extend through an entire thickness of the at least one layer.

14. The absorbent undergarment of claim 1, wherein the at least one layer is an outer most layer of the center section.

15. The absorbent undergarment of claim 1, further comprising at least a second material layer configured to control the flow of the viscous and fluid material.

16. The absorbent undergarment of claim 15, wherein the at least second layer comprises a plurality of fenestrations disposed within the at least second layer and extending at least through a portion of the at least second layer.

17. The absorbent undergarment of claim 16, wherein the plurality of fenestrations extend through an entire thickness of the at least second layer.

18. The absorbent undergarment of claim 15, further comprising a plurality of protrusions wherein each of the plurality or protrusions are separated by a space such that the flow of viscous material can be directed to and captured within the space, wherein the at least one layer of plurality of layers is further configured to absorb fluid material, and
wherein the plurality of protrusions and spaces may be configured to adjust the absorption tunability of the viscous and fluid material of the undergarment.

19. The absorbent undergarment of claim 18, further comprising a plurality of fenestrations disposed within the spaces.

20. The absorbent undergarment of claim 18, further comprising a plurality of fenestrations disposed within the protrusions.
